(19) United States
(12) Patent Application Publication
(10) Pub. No.: US 2014/0275938 A1
(43) Pub. Date: Sep. 18, 2014

(54) SYSTEM AND METHOD FOR DETERMINING REPETITIVE AIRFLOW REDUCTIONS

(71) Applicant: COVIDIEN LP, Boulder, CO (US)

(72) Inventors: Paul Stanley Addison, Edinburgh (GB); James Nicholas Watson, Dunfermline (GB)

(73) Assignee: Covidien LP, Boulder, CO (US)

(21) Appl. No.: 13/826,164

(22) Filed: Mar. 14, 2013

(51) Int. Cl. A61B 5/087; A61B 5/08

(52) U.S. Cl. CPC A61B 5/0873; A61B 5/0826
USPC 600/407

(57) ABSTRACT

Certain embodiments of the present disclosure provide a system and method for determining a repetitive airflow reduction of an individual. The system may include a photoplethysmogram (PPG) detection module configured to detect a PPG signal of a patient. The PPG signal may include a pulsatile AC component superimposed on a DC baseline. The system may also include a PPG baseline analysis module configured to analyze the DC baseline of the PPG signal to detect one or more threshold crossings with respect to an acceptable threshold correlated to normal breathing. The system may also include a repetitive airflow reduction determination module configured to determine an occurrence of the repetitive airflow reduction through an analysis of the one or more threshold crossings.

### FIELD

Embodiments of the present disclosure generally relate to physiological signal processing and, more particularly, to a system and method for determining repetitive airflow reductions through analysis of a photoplethysmographic signal.

### BACKGROUND

In various settings, a patient may be monitored for respiratory effort. For example, a breath detection device, such as a nasal thermistor, or the like, may be operatively connected to a patient. A breath waveform derived directly from breath detection may be analyzed to determine various respiratory problems, such as apnea, hypopnea, asthma, and the like. Apnea is a suspension of external breathing. Hypopnea is a disorder that involves episodes of overly shallow breathing or an abnormally low respiratory rate. Hypopnea differs from apnea in that there remains some flow of air. Typically, in order to detect respiratory afflictions such as apnea or hypopnea, the breath of an individual is directly monitored and analyzed. However, the breath of an individual may not be monitored in various clinical and medical settings. As such, respiratory afflictions may not be detected.

A pulse oximeter may be operatively connected to an individual to determine the oxygen saturation (SpO2) of blood. A photoplethysmographic (PPG) signal may be output and analyzed by an oximeter monitor. Photoplethysmography is a non-invasive, optical measurement that may be used to detect changes in blood volume within tissue, such as skin, of an individual. Photoplethysmography may be used with pulse oximeters, vascular diagnostics, and digital blood pressure detection systems. Typically, a PPG system includes a light source that is used to illuminate tissue of a patient. A photodetector is then used to measure small variations in light intensity associated with blood volume changes proximal to the illuminated tissue. As an alternative to directly measuring the breath of an individual, certain systems analyze the SpO2 signal derived from a PPG signal in order to determine reductions in airflow. However, an SpO2 signal may not always accurately indicate whether respiratory afflictions are present.

### SUMMARY

Embodiments of the present disclosure provide a system and method for analyzing a PPG baseline to determine respiratory effort in order to detect the presence of one or more respiratory afflictions, such as apnea, hypopnea, asthma, or the like. Embodiments of the present disclosure may accurately detect the presence of a respiratory affliction without directly analyzing a breath waveform of an individual.

Certain embodiments of the present disclosure provide a system for determining a repetitive airflow reduction, such as apnea, hypopnea, asthma, or the like, of an individual. The system may include a photoplethysmogram (PPG) detection module, a PPG baseline analysis module, and a repetitive airflow reduction determination module. The PPG detection module is configured to detect a PPG signal of a patient. The PPG signal may include a pulsatile AC component superimposed on a DC baseline. The PPG baseline analysis module is configured to analyze the DC baseline of the PPG signal to detect one or more threshold crossings with respect to an acceptable threshold correlated to normal breathing. The repetitive airflow reduction determination module is configured to determine an occurrence of the repetitive airflow reduction through an analysis of the one or more threshold crossings. For example, the repetitive airflow reduction determination module may be configured to determine occurrence of the repetitive airflow reduction by determining an existence of a repeating pattern of multiple threshold crossings. The system may also include a correlation module configured to correlate airflow characteristics with the DC baseline.

The repetitive airflow reduction determination module may also be configured to determine a severity of the repetitive airflow reduction through an analysis of the DC baseline. For example, the repetitive airflow reduction determination module may determine the severity based on a frequency of multiple threshold crossings during a defined time frame. Optionally, the repetitive airflow reduction determination module may determine the severity based on a time that each threshold crossing is outside of the acceptable threshold.

Certain embodiments of the present disclosure provide a method of determining a repetitive airflow reduction of an individual. The method may include detecting a PPG signal having a pulsatile AC component superimposed on a DC baseline with a PPG detection module, analyzing the DC baseline of the PPG signal, with a PPG baseline analysis module, to detect one or more threshold crossings with respect to an acceptable threshold correlated to normal breathing, and determining an occurrence of the repetitive airflow reduction with a repetitive airflow reduction determination module. The determining operation may include analyzing the one or more threshold crossings.

Certain embodiments of the present disclosure provide a tangible and non-transitory computer readable medium that includes one or more sets of instructions configured to direct a computer to detect a PPG signal having a pulsatile AC component superimposed on a DC baseline, analyze the DC baseline of the PPG signal to detect one or more threshold crossings with respect to an acceptable threshold correlated to normal breathing, and determine an occurrence of the repetitive airflow reduction through an analysis of the one or more threshold crossings.

### BRIEF DESCRIPTION OF THE DRAWINGS

FIG. 1 illustrates a simplified block diagram of a system for determining respiratory effort, according to an embodiment of the present disclosure.

FIG. 2 illustrates an airflow waveform over time, according to an embodiment of the present disclosure.

FIG. 3 illustrates a PPG waveform over time, according to an embodiment of the present disclosure.

FIG. 4 illustrates a simplified AC component of a PPG signal over time, according to an embodiment of the present disclosure.

FIG. 5 illustrates a PPG baseline having a threshold variation pattern, according to an embodiment of the present disclosure.

FIG. 6 illustrates a PPG baseline within an acceptable threshold, according to an embodiment of the present disclosure.

FIG. 7 illustrates a non-triggering PPG baseline, according to an embodiment of the present disclosure.

FIG. 8 illustrates a non-triggering PPG baseline, according to an embodiment of the present disclosure.

FIG. 9 illustrates an airflow waveform over time, according to an embodiment of the present disclosure.

FIG. 10 illustrates a PPG waveform over time, according to an embodiment of the present disclosure.

FIG. 11 illustrates an isometric view of a PPG system, according to an embodiment of the present disclosure.

FIG. 12 illustrates a simplified block diagram of a PPG system, according to an embodiment of the present disclosure.

FIG. 13 illustrates a flow chart of a method of determining a repetitive airflow reduction, according to an embodiment of the present disclosure.

### DETAILED DESCRIPTION

FIG. 1 illustrates a simplified block diagram of a system for determining respiratory effort, according to an embodiment of the present disclosure. The system 100 may include a PPG detection module 102, a PPG baseline analysis module 104, and a repetitive airflow reduction determination module 106. The system 100 may also include an airflow detection module 108 and a calibration or correlation module 110. Each of the airflow detection module 108 and the PPG detection module 102 may be in communication with the correlation module 110. Alternatively, the system 100 may not include the airflow detection module 108 and the correlation module 110.

The system 100 is configured to detect a PPG signal, such as through a PPG or pulse oximeter monitor, through the PPG detection module 102, which may be operatively connected to, and/or in communication with a detection sub-system (not shown in FIG. 1), such as pulse oximeter system. The PPG baseline analysis module 104 receives a PPG signal from the PPG detection module 102. In general, the PPG signal is a physiological signal that includes an AC physiological component related to cardiac synchronous changes in the blood volume with each heartbeat. The AC component is typically superimposed on a DC baseline that may be related to respiration, sympathetic nervous system activity, and thermoregulation. The PPG baseline analysis module 104 may filter the PPG signal to remove the AC component and determine the DC component, or baseline, of the PPG signal. Optionally, the PPG baseline analysis module 104 may not filter the AC component from the PPG signal, but, instead, analyze the DC component, or baseline, directly from the composite PPG signal. The PPG baseline analysis module 104 may analyze the DC component, or baseline, of the PPG signal, to determine whether the baseline is contained within a particular window, envelope, boundary, or other such threshold that may signal a respiratory affliction. The threshold may be an acceptable threshold correlated to normal breathing.

The repetitive airflow reduction determination module 106 then determines whether the DC component, or baseline, of the PPG signal varies with respect to the threshold, such as passing through, exceeding, being below, or otherwise crossing the threshold. The repetitive airflow reduction determination module 106 determines whether a threshold variation pattern emerges. The threshold variation pattern may include a certain number of repeating, sequential threshold variations over a certain amount of time. For example, the threshold variation pattern may include three or more dips below, or spikes above, a particular threshold over a sixty second timeframe. However, the threshold variation pattern may include more or less threshold variations over a greater or shorter timeframe. Further, the threshold variation pattern may be repeating, in the sense that a threshold crossing is followed by a certain number of intra-threshold portions, which is then followed by the same pattern one or more times. If the repetitive airflow reduction determination module 106 determines that a threshold variation pattern exists, the module 106 may generate an alert indicating a repetitive reduction in airflow, such as apnea, hypopnea, asthma, or the like. If, however, the airflow repetitive determination module determines that no threshold variation pattern exists, the module 106 refrains from generating such an alert. Additionally, once a threshold variation pattern is detected, the repetitive airflow reduction determination module 106 and/or the PPG baseline analysis module 104 may determine a severity of repetitive airflow reduction determination module 106 by analyzing the PPG baseline.

As noted, the PPG detection module 102 and the airflow detection module 108 may be in communication with a correlation module 110. The correlation module 110 may be used to correlate measured and known reductions in airflow measured with an airflow detector, such as a nasal thermistor, with baseline modulations in PPG signal detected from the PPG detection module. For example, an airflow waveform may include clear and unambiguous reductions in airflow, which are then correlated to the PPG signal detected from the PPG detection module 102. As such, certain patterns or signs of reduction in airflow may be correlated to the PPG signal. As an example, apnea shown in an airflow waveform may be correlated with distinct baseline modulations in a PPG signal. As such, when a PPG baseline exhibits the baseline modulations, or patterns, the repetitive airflow reduction determination module 106 may determine the existence of a repetitive reduction in airflow. In general, the correlation module 110 may be used to calibrate the repetitive airflow reduction determination module 106 so that the repetitive airflow reduction determination module 106 may determine the existence of a repetitive reduction in airflow through recognition of a threshold variation pattern in a PPG baseline, or DC component. The correlation module 110 may correlate the PPG signal with an airflow signal through prior clinical studies, trials, and the like.

As noted above, the system 100 may or may not include the correlation module 110 and the airflow detection module 108. Optionally, the correlation module 110 may be a separate and distinct system that correlates various repetitive airflow reduction waveforms with PPG signals. The correlated data may then be stored in the repetitive airflow reduction determination module 106.

Thus, the system 100 is configured to detect a PPG signal through the PPG detection module 102. The PPG baseline analysis module 104 analyzes the PPG baseline, or DC component, of the PPG signal to determine variance with a defined acceptable threshold that is correlated to normal breathing. The repetitive airflow reduction determination module 106 then determines whether a threshold variation pattern is present, in order to determine whether a respiratory affliction or other such repetitive reduction in airflow is present. The repetitive airflow reduction determination module 106 may be calibrated with one or more threshold variation patterns or rules that represent one or more respiratory afflictions, such as apnea, hypopnea, asthma, or the like.

The system 100 may be contained within a workstation that may be or otherwise include one or more computing devices, such as standard computer hardware. Each module 102, 104, 106, 108, and 110 may include one or more control units, such as processing devices that may include one or more microprocessors, microcontrollers, integrated circuits, memory, such as read-only and/or random access memory, and the like.

The modules 102, 104, 106, 108, and 110 may be integrated into a single module and contained within a single housing. Alternatively, each module 102, 104, 106, 108, and 110 may be its own separate and distinct module, and contained within a respective housing.

The system may also include a display 112, such as a cathode ray tube display, a flat panel display, such as a liquid crystal display (LCD), a light-emitting diode (LED) display, a plasma display, or any other type of monitor. The system 100 may be configured to show information related to repetitive reductions in airflow, such as the PPG signal, including the AC and DC components, alerts relating to a repetitive reduction in airflow, and the like, on the display 112.

The system 100 may include any suitable computer-readable media used for data storage. For example, one or more of the modules 102, 104, 106, 108, and 110 may include computer-readable media. The computer-readable media are configured to store information that may be interpreted by the modules 102, 104, 106, 108, and 110. The information may be data or may take the form of computer-executable instructions, such as software applications, that cause a microprocessor or other such control unit within the modules 102, 104, 106, 108, and 110 to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

FIG. 2 illustrates an airflow waveform 200 over time, according to an embodiment of the present disclosure. The airflow waveform 200 may be detected by an airflow detection device, such as a nasal thermistor, for example. As shown, the airflow waveform 200 includes breathing periods 202 separated by non-breathing periods 204. A healthcare professional may view the airflow waveform 200 and determine that the patient is experiencing apnea, through the clear and unambiguous non-breathing periods 204. Thus, if a patient is being monitored by an airflow detection module, respiratory afflictions may be readily discerned through the airflow waveform. However, as indicated above, the patient may not be connected to an airflow detection system, such as a nasal thermistor, or the like.

FIG. 3 illustrates a PPG waveform 300 over time, according to an embodiment of the present disclosure. The PPG waveform 300 includes an AC component superimposed on a DC component, DC baseline, or baseline 301. The AC component reflects the varying blood volume, and so optical absorption under the sensor, and is caused by the heart generating a pulsatile flow of blood through the body. The DC component (or DC baseline) is comprised of those components with constituent frequencies less than that of the cardiac pulsatile component. The DC component may be obtained by low pass filtering the PPG at just below the cardiac frequency. The AC component may be used by an oximeter to determine oxygen saturation (SpO2), for example. For example, in a pulse oximetry system, a “ratio of ratios” may be calculated by taking the natural logarithm of the ratio of the peak value of an infrared signal divided by a trough measurement of a red signal. The value is then divided by the natural logarithm of the ratio of the peak value of the red signal divided by the trough measurement of the infrared signal. In this manner, oxygen saturation may be determined.

FIG. 4 illustrates a simplified AC component 400 of a PPG signal over time, according to an embodiment of the present disclosure. Each AC component 400 pulse may represent a single heartbeat and may include a pulse-transmitted or primary peak 402 separated from a pulse-reflected or trailing peak 404 by a dichrotic notch 406. The primary peak 402 represents a local blood volume change due to the pressure wave generated at the heart and measured at a point of detection, such as in a finger, forehead, forearm, neck, or the like, where a pulse oximeter sensor, for example, is positioned. The trailing peak 404 may represent a local blood volume change due to the pressure wave that has been reflected from a location proximate to where the pulse oximeter sensor is positioned back toward the heart. Referring to FIGS. 3 and 4, the baseline 301 includes numerous AC components, such as the AC component 400, superimposed thereon.

Referring to FIGS. 2 and 3, the PPG waveform 300 may be correlated with the airflow waveform 200. In this manner, the non-breathing periods 202 may be correlated with baseline dips 302. For example, the correlation module 110 (shown in FIG. 1) may correlate the non-breathing periods 202 with the baseline dips 302. As such, the baseline dips 302 may be used to determine the existence of a repetitive reduction in airflow. For example, baseline dips 302 may be stored in the PPG baseline analysis module 104 and/or the repetitive airflow reduction determination module 106. When a repeating pattern of baseline dips 302 appears in the baseline 301, the repetitive airflow reduction determination module 106 may determine the existence of a repetitive reduction in airflow, such as apnea, hypopnea, asthma, and/or the like. A threshold variation pattern may be used to determine the existence of a repetitive reduction in airflow because a single baseline dip not followed in sequence by a repeating pattern may simply be the result of noise, interference, or motion artifacts.

FIG. 5 illustrates a PPG baseline 500 having a threshold variation pattern, according to an embodiment of the present disclosure. A baseline threshold 502 is defined with respect to the PPG baseline 500. The baseline threshold 502 may be determined through clinical and patient studies that indicate an acceptable or normal level of breathing that is correlated with the PPG baseline 500. As shown in FIG. 5, the threshold 502 spans from a peak level 504 to a lower level 506. However, the threshold 502 may be set and defined at various other levels of the PPG baseline 500.

The PPG baseline 500 includes a plurality of baseline dips 508 that drop below the lower level 506 of the baseline threshold 502. Each baseline dip 508 may indicate a reduction in breathing. However, in order to prevent false alerts, such as when a baseline dip 508 is caused by a motion artifact, for example, the repetitive airflow reduction determination module 106 (shown in FIG. 1) analyzes the PPG baseline 500 for a threshold variation pattern. The threshold variation pattern may include multiple baseline dips 508 and multiple intra-threshold portions 510. The threshold variation pattern may include alternating baseline dips 508 and intra-threshold portions 510 over a certain period of time. For example, a pattern of four or more alternating baseline dips 508 and intra-threshold portions 510 over a predefined time, such as two minutes, may trigger the repetitive airflow reduction determination module 106 to determine the existence of a repetitive reduction in airflow. However, the triggering pattern may include more or less alternations over a greater or shorter period of time.

The baseline modulations, such as caused by the threshold crossings or dips 508, may be due to changes in cardiac output through the cycle of airflow. As cardiac output increases, venous return from the peripheries may increase, and venous draining results in increased light intensity at a probe site as the local volume of blood decreases. The opposite effect occurs when cardiac output decreases.

The system 100 monitors the PPG baseline in order to determine threshold variation patterns, such as repeating patterns of repetitive threshold crossings, such as the baseline dips 508. The repetitive airflow reduction determination module 106 detects the repeating patterns of threshold crossings and correlates the patterns with repetitive reductions in airflow. Thus, instead of directly monitoring an airflow waveform, repetitive reductions in airflow may be monitored through an analysis of the PPG baseline, which may be more reliable than determining respiratory effort based on analysis of an oxygen saturation (SpO2) signal.

FIG. 6 illustrates a PPG baseline 600 within an acceptable threshold, according to an embodiment of the present disclosure. As shown in FIG. 6, the PPG baseline 600 is contained within the threshold 602. Again, the threshold 602 may be a window, envelope, or the like, that is related to the PPG baseline 600. For example, the threshold 602 may range from a peak to trough of a normal PPG baseline 600. Alternatively, the threshold 602 may be defined from various other points, such as percentage of a maximum peak and/or trough.

The PPG baseline 600 does not extend past or through upper or lower limits of the threshold 602. Accordingly, the PPG baseline 600 does not produce any baseline dips or spikes that would trigger a repetitive airflow reduction event.

FIG. 7 illustrates a non-triggering PPG baseline 700, according to an embodiment of the present disclosure. As shown in FIG. 7, the PPG baseline 700 includes s single baseline dip 701 that dips below a lower limit 703 of a threshold 702. However, in order to trigger a repetitive airflow reduction event, the PPG baseline 700 may include multiple threshold crossings, as opposed to just one. A single threshold crossing may merely be a motion artifact, noise, interference, or the like.

FIG. 8 illustrates a non-triggering PPG baseline 800, according to an embodiment of the present disclosure. As shown in FIG. 8, the PPG baseline 800 includes a first threshold crossing 802 separated from a second threshold crossing 804 by a series of intra-threshold waves 806. If the pattern from the first threshold crossing 802 to the second threshold crossing 804 does not repeat in this manner, the PPG baseline 800 may not trigger a repetitive airflow reduction event. If, however, the pattern repeats, the PPG baseline 800 may trigger a repetitive airflow reduction event.

Referring to FIGS. 5-8, triggering threshold variation patterns may be stored in the repetitive airflow reduction determination module 106. The triggering patterns may include sequential, repeating segments having multiple threshold crossings, such as baseline dips, spikes, and/or the like. For example, various patterns may be correlated with various respiratory ailments. As an example, apnea may be correlated with a pattern having a series of alternating baseline dips and intra-threshold portions. Hypopnea may be correlated with a pattern including repeating sets of a first baseline dip separated from another baseline dip by 3 intra-threshold wave portions. Each triggering patterns may be over a particular time period, such as thirty seconds, sixty seconds, or more, for example. The triggering patterns may be longer than a typical respiratory cycle, in order to disregard normal or periodic anomalies (such as a cough, sneeze, or the like) in an otherwise normal respiratory cycle.

Referring again to FIGS. 1 and 5, the repetitive airflow reduction determination module 106 may determine various degrees of respiratory afflictions through an analysis of the PPG baseline 500. For example, if the repetitive airflow reduction determination module 106 detects a triggering threshold variation pattern, such as shown in FIG. 5, the module 106 may determine the degree, severity, or seriousness of the repetitive airflow reduction determination module 106 based on an amplitude 520 of the PPG baseline 500 from peak 522 to trough 524. As the amplitude 520 increases, the seriousness of the repetitive airflow reduction may also increase. Alternatively, or additionally, the severity of the repetitive airflow reduction may be based on the time 528 the baseline dip 524 is outside of the threshold 502. The longer the time 528, the more serious the repetitive airflow reduction may be. Additionally, the repetitive airflow reduction determination module 106 may determine a degree or severity of repetitive airflow reduction based on a frequency of baseline dips 524 or crossings over a certain time frame.

Referring to FIGS. 1-8, the system 100 is configured to monitor a PPG signal. In particular, the PPG baseline analysis module 104 analyzes a PPG baseline, such as the baseline 500, to determine modulations or changes that are indicative of reductions in airflow. The repetitive airflow reduction determination module 106 may store one or more airflow reduction patterns having portions that cross a threshold in a repeating manner. The repetitive airflow reduction determination module compares the PPG baseline to the stored patterns. If the current PPG baseline matches a stored pattern, then the repetitive airflow reduction determination module 106 may determine that a repetitive airflow reduction event has been triggered, and may cause an audio or visual alert to be generated. Expected patterns of any form (for example, pattern matching) that are used to indicate reductions in airflow may use, for example, nearest neighbor methods, such as those employed by neural networks and Bayesian techniques.

Optionally, instead of recognized patterns, the repetitive airflow reduction determination module 106 may be programmed with rules to detect triggering events. For example, a triggering rule may include the presence of multiple threshold crossings separated by one or more intra-threshold wave portions. Each triggering rule may be limited to a certain period of time, for example. Each triggering rule may be correlated to a certain respiratory affliction. For example, a first triggering rule may be related to apnea, a second triggering rule may be related to hypopnea, a third triggering rule may be related to asthma, and the like. The repetitive airflow reduction determination module 106 may also determine the degree of a particular repetitive reduction in airflow through an analysis of various parameters of the baseline PPG. For example, the degree of a particular repetitive reduction in airflow may depend on the amplitude of the baseline PPG, the frequency of threshold crossings over a particular time frame, and/or the like.

The system 100 may analyze PPG baselines and determine threshold crossings and patterns through various systems and methods. For example, the PPG baseline analysis module 104 may detect baseline modulations through autocorrelation, Fourier transforms, wavelet transforms, and/or the like. In some embodiments, transforms, such as transforms corresponding to frequency or wavelet domains, may be employed. For example, transforms and operations that convert a signal or any other type of data into a spectral (i.e., frequency) domain may create a series of frequency transform values in a two-dimensional coordinate system where the two dimensions may be frequency and, for example, amplitude. As another example, transforms and operations that convert a signal or any other type of data into a time-scale domain may create a series of time-scale transform values in a three-dimensional coordinate system where the three dimensions may be scale (or characteristic frequency), time and, for example, amplitude. Wavelet transforms are further described in U.S. Pat. No. 7,944,551, entitled “Systems and Methods for a Wavelet Transform Viewer,” and U.S. Patent Application Publication No. 2010/0079279, entitled “Detecting a Signal Quality Decrease in a Measurement System,” both of which are hereby incorporated by reference in their entireties. The PPG baseline analysis module 104 may use wavelet transforms, for example, to detect dominant frequencies in the PPG baseline in order to determine the location of peaks and troughs in the PPG baseline.

The system 100 may be used in conjunction with other repetitive airflow reduction detection systems in order to provide confidence or accuracy checks. For example, the system 100 may be used in conjunction with an airflow detection system that directly detects and measures breaths of a patient in order to provide confidence metrics with respect to detection of airflow reductions. If both systems generate alerts regarding a repetitive reduction in airflow, then the confidence level is generally high.

Additionally or alternatively, the system 100 may be used in conjunction with a different system for detecting airflow reductions. For example, respiratory effort may be detected through an analysis of oxygen saturation derived from a PPG signal. While relying on an SpO2 signal to determine repetitive reductions in airflow may not always be completely reliable, the system 100 may concurrently analyze threshold variation patterns in a PPG baseline, as described above, in conjunction with an analysis of an SpO2 signal, to redundantly determine repetitive reductions in airflow. An analysis of the PPG baseline to determine repetitive reductions in airflow may indicate triggering patterns correlated with respiratory afflictions that are otherwise not capable of being determined based strictly on an analysis of an oxygen saturation signal.

FIG. 9 illustrates an airflow waveform 900 over time, according to an embodiment of the present disclosure. The airflow waveform 900 includes normal breathing segments 904, separated by reduced breathing segments 906. Unlike the airflow waveform 200, the reduced breathing segments 906 do not show complete cessation of breathing. Instead, the airflow waveform 900 represents hypopnea.

FIG. 10 illustrates a PPG waveform 1000 over time, according to an embodiment of the present disclosure. The PPG waveform 1000 may correlate with the airflow waveform 900 of FIG. 9, such that the normal breathing segments 904 correlate with PPG peaks 1002, and the reduced breathing segments 906 may correlate with PPG dips 1004. The system 100 of FIG. 1 may detect patterns and determine repetitive airflow reductions in the PPG baseline, as described above. Again, the system 100 may determine repetitive airflow reductions, such as apnea, hypopnea, asthma, and the like, through an analysis of PPG baseline. The system 100 detects repetitive patterns having repeating sequences that cross one or more thresholds of the PPG baseline in order to determine the existence of various respiratory afflictions and/or ailments.

As shown in FIG. 1, the PPG detection module 102 is configured to detect PPG signals of a patient. The PPG detection module 102 may be part of a PPG system.

FIG. 11 illustrates an isometric view of a PPG system 1110, according to an embodiment of the present disclosure. The PPG system 1110 may be in communication with, or part of, the system 100, shown in FIG. 1. For example, the PPG system 1110 may be or include the PPG detection module 102. The PPG system 1110 may be a pulse oximetry system, for example. The system 1110 may include a PPG sensor 1112 and a PPG monitor 1114. The PPG sensor 1112 may include an emitter 1116 configured to emit light into tissue of a patient. For example, the emitter 1116 may be configured to emit light at two or more wavelengths into the tissue of the patient. The PPG sensor 1112 may also include spaced-apart photodetectors 1118 that are configured to detect the emitted light from the emitter 1116 that emanates from the tissue after passing through the tissue. The photodetectors 1118 may be equidistant, but on opposite sides, from the emitter 1116.

The system 1110 may include a plurality of sensors forming a sensor array in place of the PPG sensor 1112. Each of the sensors of the sensor array may be a complementary metal oxide semiconductor (CMOS) sensor, for example. Alternatively, each sensor of the array may be a charged coupled device (CCD) sensor. In another embodiment, the sensor array may include a combination of CMOS and CCD sensors. The CCD sensor may include a photoactive region and a transmission region configured to receive and transmit, while the CMOS sensor may include an integrated circuit having an array of pixel sensors. Each pixel may include a photodetector and an active amplifier.

The emitter 1116 and the photodetectors 1118 may be configured to be located on opposite sides of a digit, such as a finger or toe, in which case the light that emanates from the tissue passes completely through the digit. The emitter 1116 and the photodetectors 1118 may be arranged so that light from the emitter 1116 penetrates the tissue and is reflected by the tissue into the detector 1118, such as a sensor designed to obtain pulse oximetry data.

The sensor 1112 or sensor array may be operatively connected to and draw power from the monitor 1114, for example. Optionally, the sensor 1112 may be wirelessly connected to the monitor 1114 and include a battery or similar power supply (not shown). The monitor 1114 may be configured to calculate physiological parameters based at least in part on data received from the sensor 1112 relating to light emission and detection. Alternatively, the calculations may be performed by and within the sensor 1112 and the result of the oximetry reading may be passed to the monitor 1114. Additionally, the monitor 1114 may include a display 1120 configured to display the physiological parameters or other information about the system 1110. The monitor 1114 may also include a speaker 1122 configured to provide an audible sound that may be used in various other embodiments, such as for example, sounding an audible alarm in the event that physiological parameters are outside a predefined normal range.

The sensor 1112, or the sensor array, may be communicatively coupled to the monitor 1114 via a cable 1124. Alternatively, a wireless transmission device (not shown) or the like may be used instead of, or in addition to, the cable 1124.

The system 1110 may also include a multi-parameter workstation 1126 operatively connected to the monitor 1114. The workstation 1126 may be or include a computing sub-system 1130, such as standard computer hardware. The computing sub-system 1130 may include one or more modules and control units, such as processing devices that may include one or more microprocessors, microcontrollers, integrated circuits, memory, such as read-only and/or random access memory, and the like. The workstation 1126 may include a display 1128, such as a cathode ray tube display, a flat panel display, such as a liquid crystal display (LCD), a light-emitting diode (LED) display, a plasma display, or any other type of monitor. The computing sub-system 1130 of the workstation 1126 may be configured to calculate physiological parameters and to show information from the monitor 1114 and from other medical monitoring devices or systems (not shown) on the display 1128. For example, the workstation 1126 may be configured to display an estimate of a patient's blood oxygen saturation generated by the monitor 1114 (referred to as an SpO₂ measurement), pulse rate information from the monitor 1114, and blood pressure from a blood pressure monitor (not shown) on the display 1128.

The monitor 1114 may be communicatively coupled to the workstation 1126 via a cable 1132 and/or 1134 that is coupled to a sensor input port or a digital communications port, respectively and/or may communicate wirelessly with the workstation 1126. Additionally, the monitor 1114 and/or workstation 1126 may be coupled to a network to enable the sharing of information with servers or other workstations. The monitor 1114 may be powered by a battery or by a conventional power source such as a wall outlet.

The system 1110 may also include a fluid delivery device 1136 that is configured to deliver fluid to a patient. The fluid delivery device 1136 may be an intravenous line, an infusion pump, any other suitable fluid delivery device, or any combination thereof that is configured to deliver fluid to a patient. The fluid delivered to a patient may be saline, plasma, blood, water, any other fluid suitable for delivery to a patient, or any combination thereof. The fluid delivery device 1136 may be configured to adjust the quantity or concentration of fluid delivered to a patient.

The fluid delivery device 1136 may be communicatively coupled to the monitor 1114 via a cable 1137 that is coupled to a digital communications port or may communicate wirelessly with the workstation 1126. Alternatively, or additionally, the fluid delivery device 1136 may be communicatively coupled to the workstation 1126 via a cable 1138 that is coupled to a digital communications port or may communicate wirelessly with the workstation 1126.

FIG. 12 illustrates a simplified block diagram of the PPG system 1110, according to an embodiment of the present disclosure. When the PPG system 1110 is a pulse oximetry system, the emitter 1116 may be configured to emit at least two wavelengths of light (for example, red and infrared) into tissue 1140 of a patient. Accordingly, the emitter 1116 may include a red light-emitting light source such as a red light-emitting diode (LED) 1144 and an infrared light-emitting light source such as an infrared LED 1146 for emitting light into the tissue 1140 at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light.

As discussed above, the PPG system 1110 is described in terms of a pulse oximetry system. However, the PPG system 1110 may be various other types of systems. For example, the PPG system 1110 may be configured to emit more or less than two wavelengths of light into the tissue 1140 of the patient. Further, the PPG system 1110 may be configured to emit wavelengths of light other than red and infrared into the tissue 1140. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the system 1110. The photodetectors 1118 may be configured to be specifically sensitive to the chosen targeted energy spectrum of the emitter 1116.

The photodetectors 1118 may be configured to detect the intensity of light at the red and infrared wavelengths. Alternatively, each sensor in the array may be configured to detect an intensity of a single wavelength. In operation, light may enter the photodetectors 1118 after passing through the tissue 1140. The photodetectors 1118 may convert the intensity of the received light into electrical signals. The light intensity may be directly related to the absorbance and/or reflectance of light in the tissue 1140. For example, when more light at a certain wavelength is absorbed or reflected, less light of that wavelength is received from the tissue by the photodetectors 1118. After converting the received light to an electrical signal, the photodetectors 1118 may send the signal to the monitor 1114, which calculates physiological parameters based on the absorption of the red and infrared wavelengths in the tissue 1140.

In an embodiment, an encoder 1142 may store information about the sensor 1112, such as sensor type (for example, whether the sensor is intended for placement on a forehead or digit) and the wavelengths of light emitted by the emitter 1116. The stored information may be used by the monitor 1114 to select appropriate algorithms, lookup tables and/or calibration coefficients stored in the monitor 1114 for calculating physiological parameters of a patient. The encoder 1142 may store or otherwise contain information specific to a patient, such as, for example, the patient's age, weight, diagnosis, and/or the like. The information may allow the monitor 1114 to determine, for example, patient-specific threshold ranges related to the patient's physiological parameter measurements, and to enable or disable additional physiological parameter algorithms. The encoder 1142 may, for instance, be a coded resistor that stores values corresponding to the type of sensor 1112 or the types of each sensor in the sensor array, the wavelengths of light emitted by emitter 1116 on each sensor of the sensor array, and/or the patient's characteristics. Optionally, the encoder 1142 may include a memory in which one or more of the following may be stored for communication to the monitor 1114: the type of the sensor 1112, the wavelengths of light emitted by emitter 1116, the particular wavelength each sensor in the sensor array is monitoring, a signal threshold for each sensor in the sensor array, any other suitable information, or any combination thereof.

Signals from the photodetectors 1118 and the encoder 1142 may be transmitted to the monitor 1114. The monitor 1114 may include a general-purpose control unit, such as a microprocessor 1148 connected to an internal bus 1150. The microprocessor 1148 may be configured to execute software, which may include an operating system and one or more applications, as part of performing the functions described herein. A read-only memory (ROM) 1152, a random access memory (RAM) 1154, user inputs 1156, the display 1120, and the speaker 1122 may also be operatively connected to the bus 1150.

The microprocessor 1148 may be operatively connected to, or include, a PPG baseline analysis module 1149, such as the PPG baseline analysis module 104 (shown in FIG. 1), and a repetitive airflow reduction determination module 1151, such as the repetitive airflow reduction determination module 106 (shown in FIG. 1). As such, the system 100 shown and described with respect to FIG. 1 may be part of, and contained within, the PPG system 1110, for example.

The RAM 1154 and the ROM 1152 are illustrated by way of example, and not limitation. Any suitable computer-readable media may be used in the system for data storage. Computer-readable media are configured to store information that may be interpreted by the microprocessor 1148. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The monitor 1114 may also include a time processing unit (TPU) 1158 configured to provide timing control signals to a light drive circuitry 1160, which may control when the emitter 1116 is illuminated and multiplexed timing for the red LED 1144 and the infrared LED 1146. The TPU 1158 may also control the gating-in of signals from the photodetectors 1118 through an amplifier 1162 and a switching circuit 1164. The signals are sampled at the proper time, depending upon which light source is illuminated. The received signals from the photodetectors 1118 may be passed through an amplifier 1166, a low pass filter 1168, and an analog-to-digital converter 1170. The digital data may then be stored in a queued serial module (QSM) 1172 (or buffer) for later downloading to RAM 1154 as QSM 1172 fills up. In an embodiment, there may be multiple separate parallel paths having amplifier 1166, filter 1168, and A/D converter 1170 for multiple light wavelengths or spectra received.

The microprocessor 1148 may be configured to determine the patient's physiological parameters, such as SpO₂ and pulse rate using various algorithms and/or look-up tables based on the value(s) of the received signals and/or data corresponding to the light received by the photodetectors 1118. The signals corresponding to information about a patient, and regarding the intensity of light emanating from the tissue 1140 over time, may be transmitted from the encoder 1142 to a decoder 1174. The transmitted signals may include, for example, encoded information relating to patient characteristics. The decoder 1174 may translate the signals to enable the microprocessor 1148 to determine the thresholds based on algorithms or look-up tables stored in the ROM 1152. The user inputs 1156 may be used to enter information about the patient, such as age, weight, height, diagnosis, medications, treatments, and so forth. The display 1120 may show a list of values that may generally apply to the patient, such as, for example, age ranges or medication families, which the user may select using the user inputs 1156.

The fluid delivery device 1136 may be communicatively coupled to the monitor 1114. The microprocessor 1148 may determine the patient's physiological parameters, such as a change or level of fluid responsiveness, and display the parameters on the display 1120. In an embodiment, the parameters determined by the microprocessor 1148 or otherwise by the monitor 1114 may be used to adjust the fluid delivered to the patient via fluid delivery device 1136.

As noted, the PPG system 1110 may be a pulse oximetry system. A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of a patient. Pulse oximeters measure and display various blood flow characteristics including, but not limited to, the oxygen saturation of hemoglobin in arterial blood.

A pulse oximeter may include a light sensor, similar to the sensor 1112, that is placed at a site on a patient, typically a fingertip, toe, forehead or earlobe, or in the case of a neonate, across a foot. The pulse oximeter may pass light using a light source through blood perfused tissue and photoelectrically sense the absorption of light in the tissue. For example, the pulse oximeter may measure the intensity of light that is received at the light sensor as a function of time. A signal representing light intensity versus time or a mathematical manipulation of this signal (for example, a scaled version thereof, a log taken thereof, a scaled version of a log taken thereof, and/or the like) may be referred to as the PPG signal. In addition, the term “PPG signal,” as used herein, may also refer to an absorption signal (for example, representing the amount of light absorbed by the tissue) or any suitable mathematical manipulation thereof. The light intensity or the amount of light absorbed may then be used to calculate the amount of the blood constituent (for example, oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

The PPG system 1110 and pulse oximetry may be further described in United States Patent Application Publication No. 2012/0053433, entitled “System and Method to Determine SpO₂ Variability and Additional Physiological Parameters to Detect Patient Status,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

FIG. 13 illustrates a flow chart of a method of determining a repetitive airflow reduction, according to an embodiment of the present disclosure. At 1300, a PPG signal is detected, such as through the use of a PPG detection module 102, as shown in FIG. 1. Next, at 1302, a PPG baseline of the PPG signal is analyzed. For example, the PPG baseline analysis module 104, as shown in FIG. 1, may analyze the baseline of a PPG signal.

At 1304, it is determined whether the PPG baseline crosses any thresholds, which may be predefined. For example, the PPG baseline analysis module 104 may determine whether threshold crossings exist. If there are no threshold crossings at 1308, the process returns to 1302. If, however, there is at least one threshold crossing, the process continues to 1306, in which it is determined whether a repeating pattern of threshold crossings exists. For example, the repetitive airflow reduction determination module 106, shown in FIG. 1, may determine whether a pattern exists based on stored patterns and/or pattern-detection rules. If no pattern exists, the process returns to 1302. If, however, a recognizable pattern exists at 1308, the process continues to 1310, in which it is determined that a repetitive reduction in airflow is present. For example, the repetitive airflow reduction determination module 106 may determine that a repetitive reduction in airflow is present based on a recognized, repeating pattern of threshold crossings. The process then returns to 1302.

Thus, embodiments of the present disclosure provide a system and method for analyzing a PPG baseline to determine respiratory effort in order to detect the presence of one or more respiratory afflictions, such as apnea, hypopnea, asthma, or the like. Embodiments of the present disclosure may accurately detect the presence of a respiratory affliction without directly analyzing a breath waveform of an individual. Embodiments of the present disclosure provide a system and method of analyzing a PPG baseline in order to detect the presence of a repeating pattern of threshold crossings, which are correlated to one or more repetitive reductions in airflow. Embodiments of the present disclosure may be used on their own to determine one or more repetitive reductions in airflow, or in conjunction with a breath analyzer, or another system configured to detect repetitive reductions in airflow as an accuracy check and/or confidence level indicator.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “module.”

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

While various spatial and directional terms, such as top, bottom, lower, mid, lateral, horizontal, vertical, front, and the like may be used to describe embodiments, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A system for determining a repetitive airflow reduction of an individual, the system comprising:
a photoplethysmogram (PPG) detection module configured to detect a PPG signal of a patient, wherein the PPG signal comprises a pulsatile AC component superimposed on a DC baseline;
a PPG baseline analysis module configured to analyze the DC baseline of the PPG signal to detect one or more threshold crossings with respect to an acceptable threshold correlated to normal breathing; and
a repetitive airflow reduction determination module configured to determine an occurrence of the repetitive airflow reduction through an analysis of the one or more threshold crossings.

2. The system of claim 1, wherein the repetitive airflow reduction determination module is configured to determine occurrence of the repetitive airflow reduction by determining an existence of a repeating pattern of multiple threshold crossings.

3. The system of claim 1, further comprising a correlation module configured to correlate airflow characteristics with the DC baseline.

4. The system of claim 1, wherein the repetitive airflow reduction determination module is further configured to determine a severity of the repetitive airflow reduction through an analysis of the DC baseline.

5. The system of claim 4, wherein the repetitive airflow reduction determination module determines the severity based on a frequency of multiple threshold crossings during a defined time frame.

6. The system of claim 4, wherein the repetitive airflow reduction determination module determines the severity based on a time that each threshold crossing is outside of the acceptable threshold.

7. The system of claim 1, wherein the repetitive airflow reduction comprises one or more of apnea, hypopnea, or asthma.

8. A method of determining a repetitive airflow reduction of an individual, the method comprising:
detecting a photoplethysmogram (PPG) signal having a pulsatile AC component superimposed on a DC baseline with a PPG detection module;
analyzing the DC baseline of the PPG signal, with a PPG baseline analysis module, to detect one or more threshold crossings with respect to an acceptable threshold correlated to normal breathing; and
determining an occurrence of the repetitive airflow reduction with a repetitive airflow reduction determination module, wherein the determining operation comprises analyzing the one or more threshold crossings.

9. The method of claim 8, wherein the determining operation comprises determining an existence of a repeating pattern of multiple threshold crossings.

10. The method of claim 8, further comprising correlating airflow characteristics with the DC baseline.

11. The method of claim 8, wherein the determining operation comprises determining a severity of the repetitive airflow reduction by analyzing the DC baseline.

12. The method of claim 11, wherein the determining a severity operation comprises determining the severity based on a frequency of multiple threshold crossings during a defined time frame.

13. The method of claim 11, wherein the determining a severity operation comprises determining the severity based on a time that each threshold crossing is outside of the acceptable threshold.

14. The method of claim 8, wherein the repetitive airflow reduction comprises one or more of apnea, hypopnea, or asthma.

15. A tangible and non-transitory computer readable medium that includes one or more sets of instructions configured to direct a computer to:
detect a photoplethysmogram (PPG) signal having a pulsatile AC component superimposed on a DC baseline;
analyze the DC baseline of the PPG signal to detect one or more threshold crossings with respect to an acceptable threshold correlated to normal breathing; and
determine an occurrence of the repetitive airflow reduction through an analysis of the one or more threshold crossings.

16. The tangible and non-transitory computer readable medium of claim 15, wherein the one or more instructions are further configured to determine an existence of a repeating pattern of multiple threshold crossings.

17. The tangible and non-transitory computer readable medium of claim 15, wherein the one or more instructions are further configured to correlate airflow characteristics with the DC baseline.

18. The tangible and non-transitory computer readable medium of claim 15, wherein the one or more instructions are further configured to determine a severity of the repetitive airflow reduction by analyzing the DC baseline.

19. The tangible and non-transitory computer readable medium of claim 18, wherein the one or more instructions are further configured to determine the severity based on a frequency of multiple threshold crossings during a defined time frame.

20. The tangible and non-transitory computer readable medium of claim 18, wherein the one or more instructions are further configured to determine the severity based on a time that each threshold crossing is outside of the acceptable threshold.
